(19) United States
(12) Patent Application Publication
(10) Pub. No.: US 2010/0256487 A1
(43) Pub. Date: Oct. 7, 2010

(54) MICRO CENTRAL LINE VASCULAR ACCESS CATHETER AND METHOD OF USE

(75) Inventors: Irvin Hawkins, Naples, FL (US); William M. Appling, Granville, NY (US)

Correspondence Address: ANGIODYNAMICS, INC., 14 PLAZA DRIVE, LATHAM, NY, 12110, US

(73) Assignee: ANGIODYNAMICS, INC., Latham, NY (US)

(21) Appl. No.: 12/754,975

(22) Filed: Apr. 6, 2010

Related U.S. Application Data
(60) Provisional application No. 61/166,803, filed on Apr. 6, 2009.

(51) Int. Cl. A61M 25/00; A61M 25/09; A61B 6/00

(52) U.S. Cl. 600/434; 600/435

(57) ABSTRACT

A vascular access catheter having a catheter shaft configured for insertion into a vein of a patient. The catheter has an exterior surface, which has a plurality of openings defined in the distal end portion of the catheter shaft, and can have respective first and second lumens. The first lumen is in fluid communication with at least one opening of the plurality of openings and is in communication with a first extension tube. The catheter has a first extension tube and a second extension tube. The first extension tube is positioned in fluid communication with the first lumen, and the second extension tube is positioned in fluid communication with the second lumen. The second extension tube is configured for high pressure CT injections, and the first extension tube is longer in length than the second extension tube.

### TECHNICAL FIELD

The present invention relates to a medical device and method, and more particularly, a catheter for use with a blood vessel.

### BACKGROUND OF THE INVENTION

Vascular access catheters provide venous access to the central circulatory system of a patient. Vascular access catheters placed for a short time period, typically less than two weeks, are called acute catheters. For those medical conditions requiring long term access to the circulatory system, chronic or permanent vascular access catheters are used. These catheters are designed to remain within the patient for indefinite periods, often more than a year. The long-term catheter is typically used for the delivery of intravenous fluids, medications such as chemotherapy drugs and antibiotics, and blood products. Venous access catheters can also be used as access mechanisms for blood sampling and the administration of contrast agents during diagnostic Computer Tomography (CT) procedures.

Vascular access catheters are placed within the central circulatory system such that the catheter tip is exposed to high blood flow rates and large blood volumes, typically in the right atrium. This placement allows for rapid delivery and dilution of drugs into the bloodstream, and thus, more effective treatment. Conventionally, permanent or chronic venous access catheters are usually placed through a subcutaneous tunnel and include dialysis catheters, central venous catheters, and peripherally inserted central catheters, also known as PICC lines.

The central venous catheter is a catheter that is typically placed into a large vein in the neck, typically an internal jugular vein, or a subclavian vein and is used to administer medication or fluids, obtain blood tests and directly obtain cardiovascular measurements, such as, for example, the central venous pressure. The CVC allows certain medications to be given through a central line. Central venous catheters (CVCs) are larger in diameter than PICC lines and are typically placed directly into the jugular vein when no peripheral veins are available, when medications cannot be introduced into a peripheral vein without vessel damage, or when central venous pressure monitoring is required. Typically, CVCs are not used for power or CT injections. CVCs have some disadvantages that include risks associated with insertion and catheter care and potential sepsis, occlusion, infection of the line site, and accidental removal problems. If a CVC is inserted into the jugular vein, air embolisms may occur. An air embolism results when air enters the vasculature, and it can occur during the insertion or removal of central venous catheters. An air embolism may be caused when small amounts of air can get into the blood circulation accidentally during surgical procedures. It can occur whenever there is a sub-atmospheric pressure in the venous system at a point where it is open to the atmosphere. These conditions are most likely to exist in a patient in whom the central venous pressure is low and/or the fluctuations in intrathoracic pressure are large. Such conditions could include jugular venipuncture of patients sitting in the semi-upright or upright position. For air to enter the venous circulation, there must be both a direct communication between the atmosphere and a non-collapsed vein and a pressure gradient favoring the passage of air into the circulation. Air embolisms are dangerous because they rarely show symptoms and can result in death if a bubble of gas becomes lodged in the heart, stopping blood from flowing from the right ventricle to the lungs.

PICC lines, which are smaller in diameter that CVC lines, are typically introduced through the basilic, median cubital, cephalic, or brachial veins of the arm into the central circulatory system. These options, however, are disadvantageous because the veins can be small and difficult to access. In other aspects, such as in the case of the basilic vein, the insertion site can be too far medially for insertion or routine care, or the practitioner can only be able to palpate a short segment of the vessel. In the case of the median cubital vein, the patient's anatomy can vary greatly from patient to patient, or it can be difficult to insert due to valve location within the vein. If the PICC is inserted into the cephalic vein, the vessel can be small and tortuous, and the angle at which the vein joins the auxiliary vein can make it difficult to advance the catheter. If the PICC is inserted into the brachial vein, the deep location makes insertion without ultrasound difficult, or the proximity to the median nerve can pose a risk of nerve injury. Although traditionally PICC lines have been inserted directly into the target vein, placing PICC lines through a subcutaneous tunnel is an emerging trend. For these lines, the tunnel is usually created in the lower or upper arm region, with the catheter entering the target vein near the shoulder. It is important that the catheter be placed so as to eliminate any dislodgement or movement of the catheter during long-term use. Catheter movement can result in sub-optimal treatment due to catheter tip misalignment, catheter leakage, infection and blood loss. Dislodgement of a vascular access catheter can even result in death, due to extensive blood loss from the jugular or other large vein.

In operation, PICCs are typically inserted using one of three conventional insertion techniques: 1) over the needle peel-away sheath direct approach, after which the needle is removed from the peel-away sheath and the catheter is advanced through the sheath; 2) short wire introduction with a sheath dilator combination for catheter advancement (modified Seldinger technique); and an 3) over-the-wire Seldinger technique requiring fluoroscopic guidance for wire advancement into the SVC. PICCs are typically placed such that the tip of the catheter terminates in the lower third of the Superior Vena Cava (SVC) above the right atrium of the heart.

PICCs are commonly used for diagnostic imaging, for instance, injecting into a blood vessel a dye, or contrast media, that is visible under computer tomographic (CT) imaging. CT imaging is commonly used for diagnostic purposes to provide clear images of bone, muscle, and blood vessels, for example, during angiography or other procedures. CT injectors are used in CT imaging to inject a controlled volume of contrast media or dye through the catheter and into a desired location of a blood vessel at a high pressure. When the dye is injected into the blood vessel, it mixes with the blood, which makes the blood less permeable by x-ray and allows the vasculature to be visualized. This also allows direct access to the flowing blood. The images that are obtained show the outline of the blood vessel where the dye flows. Typically, before the dye is injected, the catheter is flushed with a solution such as saline to ensure that the catheter is not occluded or damaged.

PICCs or central lines are beneficial because performing CT injections through an existing PICC or central line that is already in place inside of a blood vessel avoids the need for an additional access site. The ability to inject a dye through the catheter that is already in place saves time, money, and the patient from another medical procedure to place a catheter or needle only for dye injections. Further, contrast injections through central lines deliver contrast to the central circulation, thereby providing better mixing, higher concentrations, and better images with less total contrast delivered. Additionally, there are no shearing forces from the injection being applied to the walls of small veins.

While PICCs have some advantages, they also have some disadvantages. For example, patients with PICC lines are often very ill, and gaining access to a vein is often difficult for the caregiver as well as being painful and traumatic for the patient. Continuous access of the venous system by IV needles or catheters can result in eventual destruction of the available veins. As a result of the limitations of conventional PICC lines, the physician needs to gain separate access with an IV-type needle. Conventionally, a needle is placed in the forearm area and is used to inject contrast media during the diagnostic CT procedure. This separate access site increases the complexity and time of the diagnostic procedure in addition to increasing the risk associated with the separate access site, such as bleeding, hematomas, infection, and extravasation.

PICCs are disadvantageous because they can be difficult to use and bulky, they can require multiple sticks into the patient, and they can involve lengthy procedure times. Additionally, PICC insertion procedures can cause additional pain and/or trauma to the patient because of the repeated intrusion that is required to enter a small vein, for example, the small antecubital vein. One will appreciate that this is especially problematic in obese patients, even if they have useable veins.

PICCs also pose a problem because they typically have very long catheter shafts that occupy large portions of a patient's vessel, and they have small internal diameters that permit flow rates of only 5 cc/sec, which is sub-optimal for procedures which require high flow rates, such as, for example, pheresis and CTA (computed tomography angiography). The insertion of the long catheter shaft into very small veins can also result in blood clotting and destruction of the veins, including the subclavian vein. PICCs also pose a disadvantage because PICCs can migrate after insertion; can be subject to thrombosis; can become blocked, which makes it difficult to flush or aspirate the catheter, or can be prone to infection around the insertion site or in the vein. PICCs also have a higher incidence of catheter malfunction and a higher complication rate when compared to conventional central venous catheters (CVCs). Further, non-central positioning of the catheter tips of PICCs are a very common problem. These complications tend to accrue with multiple attempts at placing the PICC, in addition to the frequent need to place multiple PICCs due to PICC failures.

What is needed is a vascular access catheter that overcomes the deficiencies of a standard PICC, yet still has all of the advantages of a standard PICC, including the capability of withstanding high pressure CT injections. What is also needed is a catheter that can be used like a central venous catheter (CVC), including insertion into the jugular vein, with minimal procedure time, patient discomfort, and the chance of complications, thereby providing the benefits of both types of catheters.

### SUMMARY

A vascular access catheter is provided that has a catheter shaft that is configured for insertion into a vein of a patient. In one aspect, the catheter has an exterior surface that extends between a proximal end and a distal end that defines a plurality of openings that are positioned in the distal end portion of the catheter shaft. In a further aspect, the catheter also has a first lumen extending from the proximal end to the distal end portion of the catheter shaft that is in fluid communication with at least one opening of the plurality of openings. Optionally, the catheter can have a second lumen that extends from the proximal end to the distal end portion of the catheter shaft. In yet another aspect, the catheter has a first extension tube and second extension tube that are positioned in fluid communication with the first lumen and second lumen, respectively. The first extension tube is longer in length than the second extension tube. The second extension tube is configured for high pressure CT injections.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed. Related methods of operation are also provided. Other devices, methods, features, and advantages of the vascular access catheter will be or become apparent to one with skill in the art upon examination of the following figures and detailed description. It is intended that all such additional devices, methods, features, and advantages be included within this description, be within the scope of the vascular access catheter, and be protected by the accompanying claims.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 illustrates a plan view of a micro central line vascular access catheter.

FIG. 2 illustrates a sectional view of the distal portion of the vascular access catheter of FIG. 1.

FIG. 2A illustrates a cross-sectional view of the catheter along line 2A-2A of the catheter shaft.

FIG. 2B illustrates a cross-sectional view of the catheter along line 2B-2B of the catheter shaft.

FIG. 3 illustrates a sectional view of the distal portion of a single lumen embodiment of the catheter shaft.

FIG. 4 illustrates a schematic view of the micro central line vascular access catheter after a distal end potion of the catheter has been inserted into a patient's jugular vein.

FIG. 5A illustrates a schematic view of the catheter of FIGS. 1-4 showing a distal end portion of the catheter inserted into a patient's jugular vein.

FIG. 5B illustrates a partial exploded view of the catheter hub of the catheter of FIGS. 1-4.

FIG. 6 illustrates a plan view of an alternative embodiment of the hub of the catheter.

FIG. 6A illustrates an exploded sectional view of the hub of the catheter of FIG. 6.

FIGS. 7A through 7D illustrate an exemplary method of inserting the catheter of FIGS. 1-4 into a patient's jugular vein.

FIG. 8 is a plan view of an additional embodiment of the micro central line vascular access catheter.

FIG. 9A is a plan view of the micro central line vascular access catheter of FIG. 8 with a valve positioned in the hub of the catheter and a guide wire inserted into one of the lumens of the catheter.

FIG. 9B is an exploded sectional view of the hub of the micro central line vascular access catheter of FIG. 9A.

FIG. 10A is plan view of the micro central line vascular access catheter of FIG. 8 with a guide wire inserted into one of the two lumens of the catheter.

FIG. 10B is a plan view of the micro central line vascular access catheter of FIG. 8 with a guide wire inserted into the single lumen of the catheter.

### DETAILED DESCRIPTION OF THE INVENTION

The present invention can be understood more readily by reference to the following detailed description and the examples included therein and to the Figures and their previous and following description. The drawings, which are not necessarily to scale, depict selected preferred embodiments and are not intended to limit the scope of the invention. The detailed description illustrates by way of example, not by way of limitation, the principles of the invention.

The skilled artisan will readily appreciate that the devices and methods described herein are merely exemplary and that variations can be made without departing from the spirit and scope of the invention. It is also to be understood that the terminology used herein is for the purpose of describing particular embodiments only and is not intended to be limiting.

Ranges can be expressed herein as from “about” to one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. As used herein, the terms “outwardly” and “inwardly” designate directions in the drawings to which reference is made. The terms “proximal” and “distal” refer to directions away from and closer to, respectively, the insertion tip of the catheter disclosed herein. The terminology includes the words above specifically mentioned, derivatives thereof, and words of similar import.

“Optional” or “optionally” means that the subsequently described element, event or circumstance can or can not occur, and that the description includes instances where said element, event or circumstance occurs and instances where it does not.

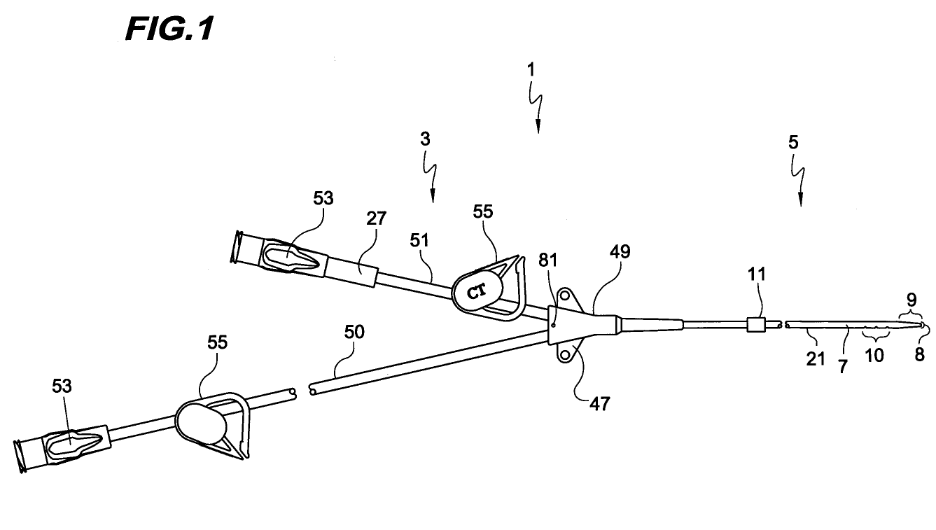

Referring now in detail to the drawings, in which like reference numerals indicate like parts or elements throughout the several views, in various embodiments, and referring to FIGS. 1 through 10B, presented herein is an exemplary vascular access catheter, such as a micro central line vascular access catheter, a method of inserting the catheter into the patient's body, and a method of injecting infusates or contrast agents under high pressure or CT injections into the vascular access catheter through a CT injection lumen of the vascular access catheter.

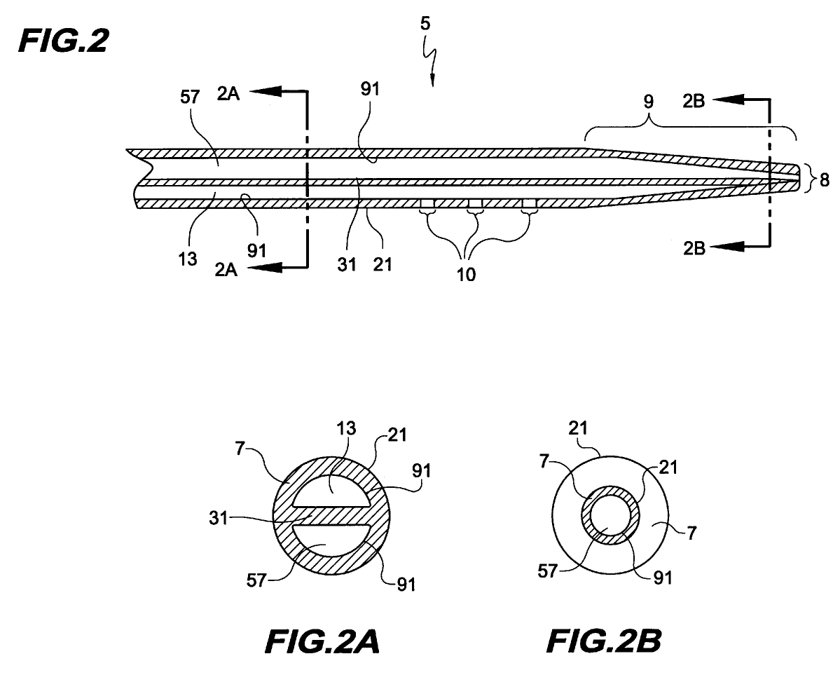

FIGS. 1 and 2 illustrate one embodiment of a micro central line vascular access catheter. In one aspect, the catheter 1 has a proximal portion 3 and a distal portion 5. At least a portion of the catheter shaft 7 forms the distal portion 5 of the catheter 1. In one aspect, the proximal portion 3 of the catheter 1 can be comprised of at least one of a bifurcate or hub 49 and suture wings 47, which can be coaxially arranged around the proximal portion of the bifurcate 49, extension tubes 50 and 51, extension tube clamps 55, and catheter hub connectors or luers 53.

In one exemplary aspect, the leg clamps 55 are coaxially arranged around the extension legs 50, 51 and can be used to clamp off or occlude the catheter lumens, preventing inflow or outflow of fluids through the catheter 1 when the catheter is not in use. In this aspect, at least a portion of the distal portion 5 of the catheter is formed by a catheter shaft 7 that extends from the bifurcate 49 to a distal tip 8 at the distal most end of the distal portion 5 of the catheter shaft 7.

In one aspect, the hub 49 has an exterior surface and further defines an opening 81 in the exterior surface of the hub 49. In another aspect, the catheter shaft 7 has an outer wall 21 that has at least one opening 10. In one aspect, and not meant to be limiting, the catheter 1 can have a cuff 11 that is coaxially mounted on the catheter shaft 7. Catheter cuff 11 can be positioned along the length of the catheter 1 to stabilize the catheter position when implanted in tissue. The cuff 11 can help prevent the ingrowth of tissue and can prevent infectious agents from migrating along the length of the catheter into a patient's body.

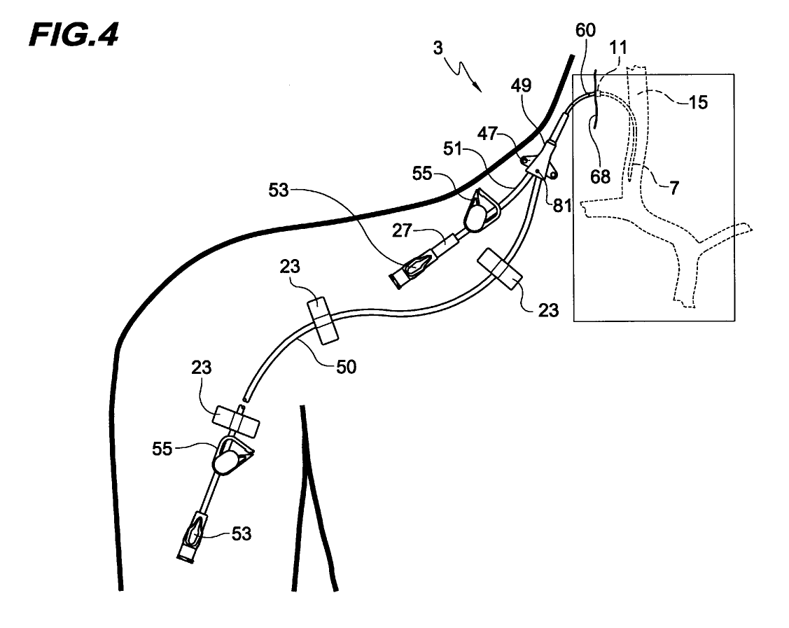

In various exemplary aspects, the outer diameter of the extension tubes 50, 51 can be approximately 0.100 inches in diameter, and the inner diameter of the extension tubes 50, 51 can be approximately 0.053 inches in diameter. In one exemplary aspect, the outer diameter of the extension tubes 50, 51 may be as great as approximately 0.50 inches. As illustrated in FIGS. 1 and 4, the extension tubes 50, 51 may extend outwardly from the hub 49 of the catheter. In another exemplary aspect, the extension tubes 50, 51 may have a pre-curved configuration, such that they curve away from the insertion site. In another aspect, the catheter shaft 7 can be a 4 or 5 French catheter shaft. In one aspect, the catheter shaft can be a 5 French dual lumen catheter with an outer diameter of approximately 0.066 inches. As one skilled in the art will appreciate, it is contemplated that other diameter catheters are within the scope of this invention.

In one aspect, as illustrated in FIGS. 1 and 2, at least a portion of the distal end 5 of the catheter 1 can be tapered. For example and without limitation, the outer diameter of the catheter shaft 7 tapers from an outer diameter of approximately 0.066 inches to an outer diameter of approximately 0.053 inches at the distal most edge of the tapered tip 8. In one aspect, the catheter shaft 7 has an inner diameter of approximately 0.022 inches at the distal most edge of the tapered tip 8 of tapered portion 9, such that it is configured for selective receipt of an 0.018 inch guide wire. As illustrated in FIG. 2, at a point distal of the distal most aperture 10, the outer wall 21 of the catheter shaft 7 begins to taper inward toward a horizontal axis of the catheter shaft 7. The catheter 1 tapers from a double D configuration at the proximal end of the catheter to a single lumen configuration at the distal most portion of the catheter tip 8. In this aspect, it is contemplated that the tapered portion 9 tapers toward the distal most portion of the catheter shaft 7.

Conversely, in one aspect, the outer wall 21 of the catheter shaft 7 can increase in thickness toward the inner lumen 57 along the tapered section 9 towards the distal end 5 of the catheter 1. In one non-limiting example, the outer wall 21 of the catheter shaft 7 tapers at an angle of approximately 10 degrees relative to the longitudinal axis of the catheter shaft 7. In one aspect, the tapered portion 9 of the catheter 1 is approximately 1 cm in length, although the length can vary based on the angle of the tapered portion. The term “tapered,” as it pertains to the description herein, means that at least a portion of the distal most end portion of the catheter 7 has an angled edge that is angled inwardly toward the longitudinal axis of the catheter and is not positioned at a perpendicular angle or in a parallel manner relative to the longitudinal axis of the catheter 1. In this aspect, the tapered distal end forms a self-dilating, atraumatic distal tip that can prevent trauma to the inside of the vessel wall.

As illustrated in FIG. 2, in one aspect, at least one port 10 can optionally be defined in the outer wall 21 of the catheter shaft 7. The at least one port 10 is in fluid communication with the first lumen 13 and the exterior of the outer wall 21 of the catheter shaft 7. In one aspect, the exterior surface 21 of the catheter shaft 7 has a plurality of side holes, openings, or apertures 10 defined therein the exterior surface 21 of the catheter shaft 7. In one example, the at least one port 10 can be defined in the exterior surface 21 of the catheter shaft 7 and can be spaced proximally from the distal tip or aperture 8. In various exemplary aspects, the port(s) 10 can be positioned along the catheter shaft 7 anywhere from the distal tip 8 of the catheter 1. In another aspect, the apertures 10 can be approximately 0.025 inches in diameter. It is contemplated that the apertures 10 can be evenly spaced along the catheter shaft 7, or they can be spaced apart in any desired manner.

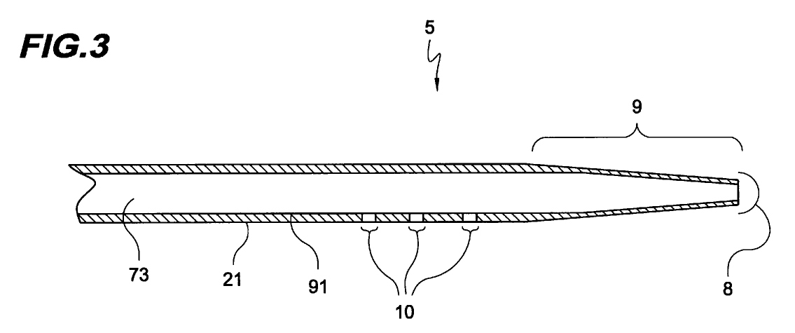

In the dual lumen embodiment illustrated in FIGS. 1 and 2, in one aspect, the at least one port 10 is defined in a sidewall of the distal end portion of the catheter shaft 7 in the first lumen 13 in the outer wall 21 of the catheter shaft 7. Optionally, such as, for example, in the single lumen embodiment illustrated in FIG. 3, the at least one port 10 can be defined thereon the catheter shaft 7 at any desired position on the catheter shaft 7. The at least one port 10 allows for the efficient infusion of infusates, such as drugs, into lumen 13 or 73 and through the at least one port 10 to dissolve fibrin sheaths or other occlusive material that has the tendency to form along the catheter shaft 7 after the catheter 1 has been implanted in a patient body, as described further herein.

In one aspect, as illustrated in the cross-sectional views along lines 2A-2A and 2B-2B, respectively, the outer wall 21 of the catheter shaft 7 surrounds the first lumen 13 and the second lumen 57, which are separated by an internal septum 31. In one aspect, the first lumen 13 and second lumen 57 can have a D-shaped lumen configuration, as illustrated along line 2A-2A in FIG. 2. Of course, it is contemplated that the lumens of the catheter 1 can have any suitable cross-sectional lumen shape as required for the particular use of the catheter 1. In one example, and not to be limiting, the internal septum 31 can have a width of approximately 0.020 inches, and the height of each double-D lumen 13 and 57 can be approximately 0.046 inches.

In one aspect, the first lumen 13 extends longitudinally from the proximal most end of the catheter shaft 7 through a partial length of the catheter shaft 7 and terminates distally of the distal most aperture 10 where the internal septum 31 merges with the inner wall 91 of the catheter shaft 7. In one aspect, as illustrated along line 2B-2B of FIG. 2, the catheter shaft 7 transitions to a single lumen 57 distal of the portion where the internal septum 31 joins the inner wall 91 of the catheter 1. Thus, in one aspect, the second lumen 57 extends longitudinally from the proximal end 3 of the catheter shaft 7 to the distal tip 8 of the shaft 7. In one aspect, the distal tip 8 can have an atraumatic, self-dilating, tapered leading edge that helps to minimize the trauma to the vessel of the patient.

In one aspect, first extension tube 50 can be in fluid communication with first lumen 13, as illustrated in FIGS. 1 and 2, or it can be in fluid communication with lumen 73 of a single lumen catheter, as illustrated in FIG. 3, and second lumen 57 is in fluid communication with second extension tube 51. In one aspect, the single lumen catheter embodiment can be used for injection of contrast agents or chemotherapeutic agents, or for blood draws, for example. In one aspect, the single lumen 73 can be in fluid communication with either extension tube 50 or 51. Both first and second extension tubes 50, 51 communicate with lumens 13, 57 through bifurcate 49. It is contemplated that any of extension tubes 50, 51 can have an identification (ID) tag 27 denoting the flow rate and/or volume of fluid to be injected into the catheter 1.

This micro central line catheter's first elongate extension tube 50 is beneficial because it can be used for blood draws during acute treatment. The first extension tube 50 is longer in length, compared to the second extension tube 51, i.e., the first extension tube has an elongate length that is greater than the second extension tube. The first extension tube 50 extends proximally past the second extension tube 51. The second extension tube 51 is beneficial for high pressure CT injections because it can inject a higher bolus concentration of contrast agent at a higher density compared to typical CT extension tube lumens. This allows the amount of contrast medium to be conserved.

As illustrated in FIG. 4, after the catheter 1 is inserted into a patient, the first extension tube 50 is configured to extend from the jugular vein 15 to the middle of the patient's arm. The first extension tube 50 is placed away from the insertion site 68, ideally at the anecubital fossa or elbow (not shown). The extension tube 50 is then secured to the patient's arm. In one aspect, fixation tabs 23 can be used to secure the extension tube 50 to the patient's arm. The securement of the first extension tube 50 to the patient's arm provides familiarity for medical practitioners who are accustomed to using PICCs that are typically secured to the patient's arm in the same location. The placement of the longer first extension tube 50 along a patient's arm is also beneficial because it reduces the infection rate at the entrance or incision site 68 where the catheter 1 is inserted.

The shorter second extension tube 51 is beneficial because it can be used to provide optimally high flow rates for such procedures as dialysis, pheresis, high pressure CT injections, and CTA (computed tomography angiography), to name a few. In one aspect, the second extension tube 51 can be capable of handling flow rates of approximately 25 cc/sec. The second extension tube 51 can also be used to aspirate and flush fluids. Second extension tube 51 allows for higher injection and flow rates, compared to first tube 50 because it is a shorter elongate length as compared to the first extension tube 50 and has a shorter lumen. In one aspect, second extension tube 51 can have a wider diameter than first extension tube 50 that can allow for higher pressures and higher flow rates.

As exemplarily configured, the shorter, wider second extension tube 51 allows fluid to be injected at high flow rates, which is especially beneficial during CT injections. In one non-limiting example, the length of the CT injection lumen of the extension tube 51 can be approximately one-third the length of the longer extension tube 50 catheter shaft 7 that is used for blood draws, which is beneficial because it allows the lumen of the second extension tube 51 to be positioned near the heart to allow for higher flow rates. In the catheter 1 disclosed herein, the diameter of the second extension/CT tube 51 can be the same diameter as traditional PICC lines, which allows for higher pressures and a flow rate that is three times as fast through the CT extension tube as it is for the blood draw extension tube 50. For instance, and not meant to be limiting, the flow rate of the blood draw lumen of extension tube 50 of catheter 1 can be capable of a 5 cc/sec flow rate, while the CT injection lumen of the CT extension tube 51 can be capable of a 15 cc/sec flow rate or more if the CT lumen is larger than the blood draw lumen. This is beneficial because it allows contrast solution to be injected at a faster rate, and potentially under higher pressures than normal through the CT injection lumen of standard PICC lines.

In one alternative aspect, the catheter 1 may be connected to a machine for dialysis or to an injection or aspiration device. In this aspect, blood can be withdrawn from the vessel of the patient into lumen 57 where it is passed through the second extension tube 51 and into a dialysis machine. Blood can be returned to the patient through first extension tube 50 into lumen 13, which exits through the distal apertures 10 into the vessel of the patient. One skilled in the art will appreciate that, although designated herein as first and second lumens 13 and 57, respectively, dialysis can be performed by reversing the blood flow through the lumens. Hence, the terms first lumen and second lumen can also be used herein to designate the interchangeability of the first and second lumens, respectively.

In one aspect, at least a portion of the outer surface of the respective extension tubes 50, 51 can be coaxially coated with an elastomeric material such as a Silastic® silicone rubber material or urethane to prevent kinking, to allow for increased flow rates, increased softness and comfort for the patient, and to provide better durability. In one additional aspect, the separation of the respective extension tubes 50, 51 prevents cross-contamination between the two extension tube lumens.

In another aspect, extension tubes 50, 51 can have glued valves (not shown) attached to the catheter hub connectors 53 which can only be opened when engaged with a luer lock syringe to prevent air embolization and blood loss. It is contemplated that fluid(s) can be introduced through the injection valves. This is beneficial because if standard one way valves are used, and such a valve is inadvertently left opened, the patient can lose blood, or air could be aspirated therein a line. It is contemplated that the luer lock on this catheter 1 can be disconnected as the valve is closed. The luer lock valves attached to the two lumens of the extension tubes of the PICC prevent the catheter from bleeding or from drawing in air during routine blood draws or during a CTA procedure.

In one non-limiting example, the useable length of the catheter shaft 7 can be between approximately 10 cm to 15 cm, depending on the patient's anatomy and physician preference. The term “useable length”, as defined herein, means the length from the distal edge of the cuff 11 of the catheter to the distal tip 8 of the catheter. In one aspect, the catheter 1 can be a 4 or 5 French catheter, although, as one skilled in the art will appreciate, any suitable size catheter can be used. The diameter and length of the catheter 1 is configured for insertion into the jugular vein of a patient, although any vein of the patient may be used. In this aspect, the diameter of the catheter is small compared to the diameter of the jugular vein so that the risk of damaging the vessel or causing patient discomfort is minimized. In one aspect, the shorter length of the catheter shaft 7, as compared to conventional catheter lengths, is beneficial because it allows for high flow rates.

In one aspect, the catheter 1 is a unitary catheter composed of Carbothane® thermoplastic polyurethane, but any suitable material can be used, such as, but not limited to, other types of polyurethane or silicone. In another aspect, the catheter 1 and the extension tubes 50, 51 can be made of any material, such as elastomeric materials or other high pressure types of tubing, that are suitable for high pressure injections or CT injections or other functions for which angiographic catheters can be used. In another aspect, the catheter 1 can also contain a radiopaque material to enhance visibility under fluoroscopy. In one aspect, the catheter may have a catheter shaft in which the durometer and radiopaque material are both varied throughout the catheter shaft, as described in co-pending U.S. Pat. No. 7,618,411, which is incorporated herein by reference.

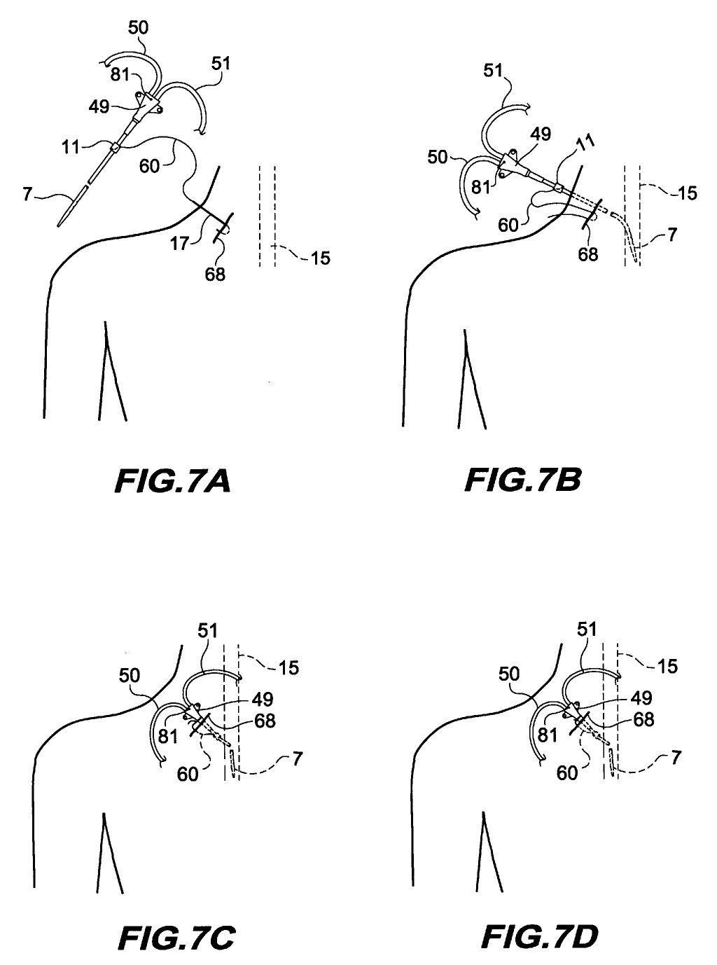

As illustrated in FIGS. 4 through 7D, disclosed herein is a method of inserting the micro central line access catheter 1 into a patient's vessel. FIG. 4 illustrates the insertion of one embodiment of the catheter 1 into a patient. After the catheter is inserted, as described below and illustrated in FIGS. 7A-D, the catheter may then be attached to the patient's skin using the suture wings 47, as is known in the art. Alternatively, as illustrated in FIG. 4, in one aspect, after the catheter 1 is inserted into the patient, a suture 60 can be tied around the catheter shaft 7 after the catheter 1 has been inserted into the patient. In another aspect, the suture thread 60 or wire can be sewn directly through the skin around the hub 49 of the catheter shaft 7 without the use of a suture wing 47, after insertion of the catheter 1.

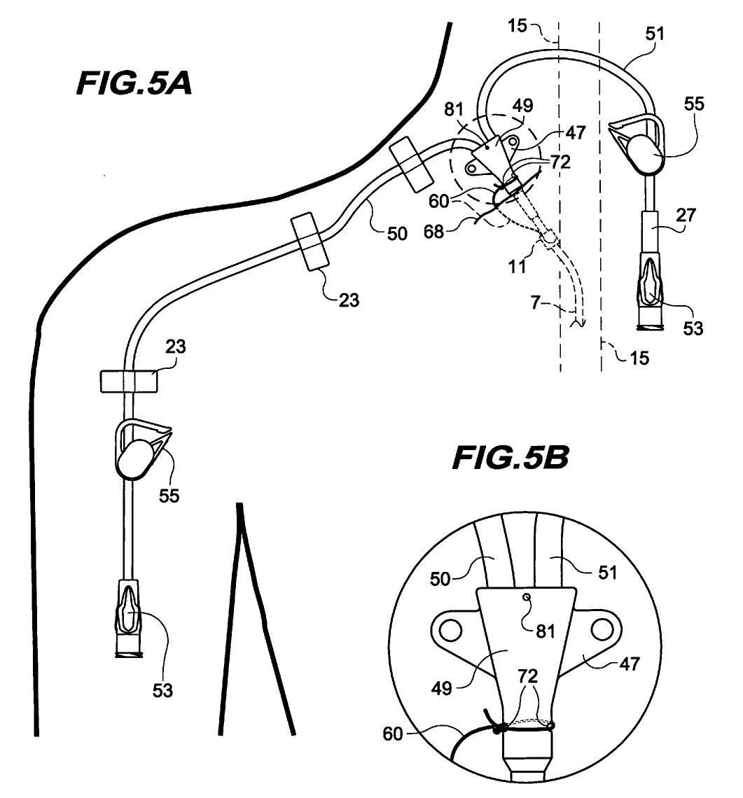

In one aspect, to prevent possible needle puncture of the catheter shaft 7, the suture 60 can be pre-attached underneath the cuff 11 to at least a portion of an inner surface of the cuff 11 and secured around the bifurcate 49, after the catheter 1 is inserted into the patient, as illustrated in FIGS. 5A and 7C-7D. In one aspect, the bifurcate 49 can have at least one groove 72 defined therein the exterior surface of the hub 49 on opposing sides of the hub 49 that is capable of receiving the suture 60, as illustrated in FIGS. 5A and 5B. In one aspect, the hub 49 can comprise a plurality of grooves 72 defined therein the exterior surface of the hub 49.

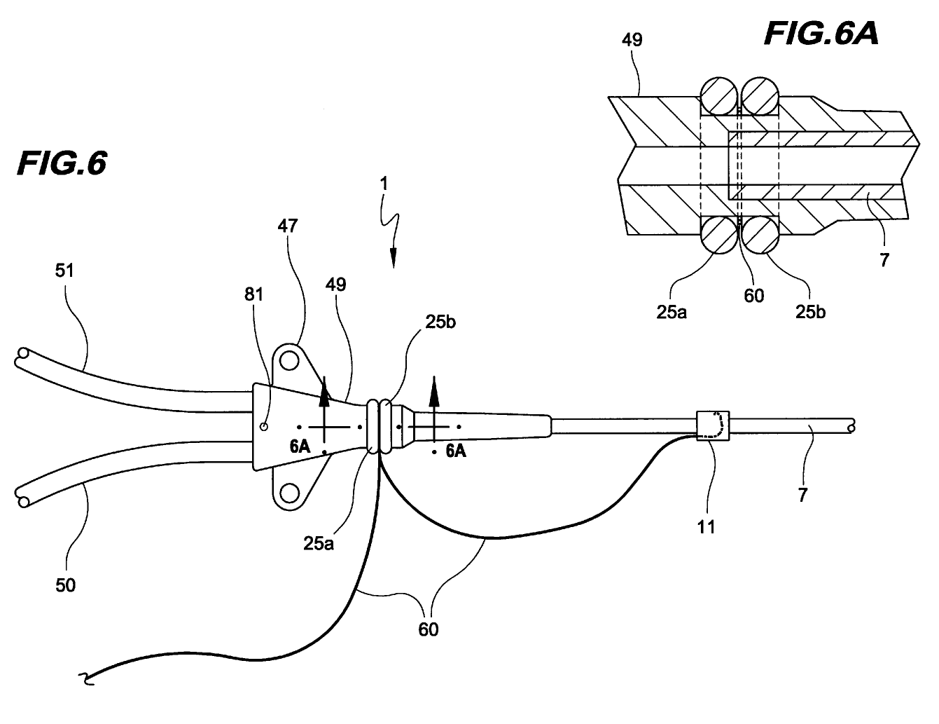

An alternative embodiment of the hub 49 is illustrated in FIGS. 6 and 6A. In one aspect, at least two rings 25a and 25b can substantially circumferentially surround at least a portion of the exterior surface of the hub 49 of the catheter shaft 7. In one exemplary aspect, the rings 25a, 25b can be composed of silicone. In another aspect, the rings can be composed of any suitable elastomeric material. The rings 25a and 25b can be positioned substantially adjacent to one another. In one exemplary aspect, the rings 25a, 25b may be in contact with each other. The rings 25a, 25b can be positioned next to each other for selective receipt of a suture 60. The rings 25a and 25b can be positioned such that when a suture 60 is inserted into and wrapped between the rings 25a, 25b, the rings self-seal, thereby securing the suture 60 between the rings 25a, 25b. In one aspect, the silicone rings 25a and 25b may be of a different color than the hub 49 of the catheter. In one aspect, the rings 25a, 25b can be blue in color, for example, so as to provide a contrast to the hub 49 of the catheter 1. In one exemplary aspect, the outer diameter of the rings 25a, 25b can be approximately 0.190 inches, and the inner diameter of the rings 25a, 25b can be approximately 0.078 inches.

As illustrated in FIGS. 5A, 5B and 7A-D, the pre-attached suture 60 that is positioned underneath the cuff 11 can: 1) help prevent the cuff 11 from sliding out or retracting out of the tissue track in which the catheter 1 is inserted, 2) help to prevent inadvertent dislodgement until tissue grows into the cuff 11, and 3) eliminate the need to replace the catheter 1. In one aspect, the suture thread 60 can be a large bio-absorbable suture thread, or it can be capable of being removed by a medical practitioner during treatment. A bio-absorbable suture thread is beneficial compared to standard removable sutures because standard sutures can become infected during the course of catheter implantation.

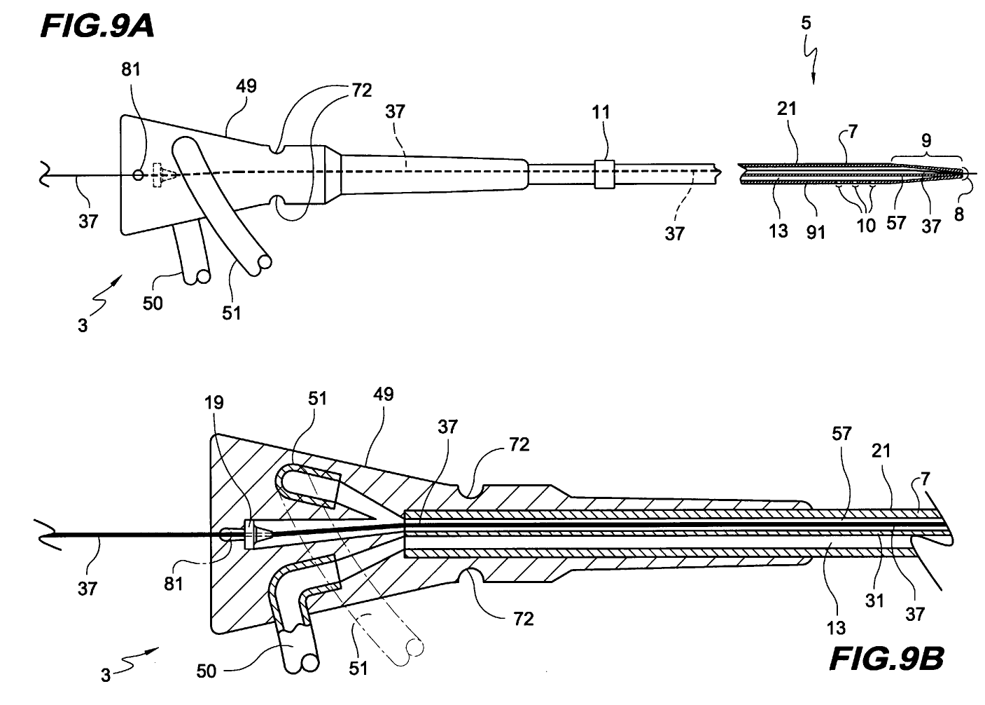

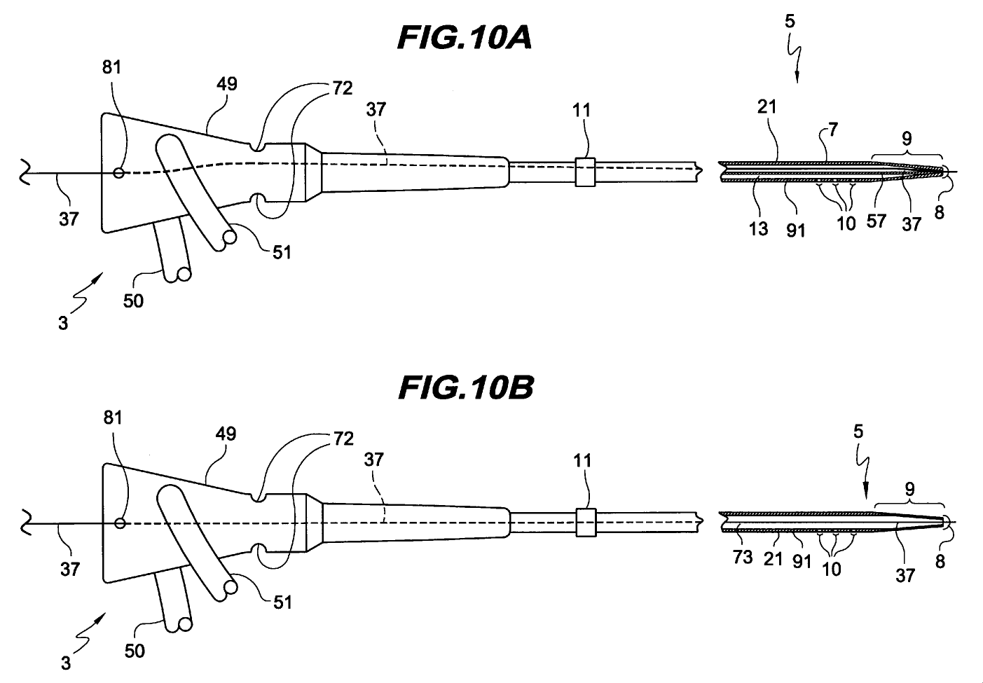

Before insertion of the catheter 1, as is known in the art, the skin is cleaned, and local anesthetic is applied, if required. As illustrated in FIG. 7A, the pre-attached suture 60 is threaded into the patient's skin using, for example, a hooked needle 17. The location of the vein into which the catheter 1 will be inserted can be identified by landmarks or with the use of ultrasound. The patient's skin can be stuck once with a hollow needle (not shown) at incision site 68. The hollow needle is advanced through the skin until blood is aspirated. A blunt guide wire is passed through the needle, and the needle is then removed. The catheter shaft 7 is then inserted into the insertion site 68 over a guide wire 37 (illustrated in FIGS. 9A-9B and 10A-10B) under ultrasound and tunneled underneath the skin into the jugular vein 15, as illustrated in FIG. 7B using a tunnel track. In one aspect, the tunnel track can be approximately 1 cm in length. This length of the tunnel track can be shorter compared to typical tunnel tracks because the catheter shaft 7 is shorter compared to typical catheters.

The catheter shaft 7 can be advanced into the jugular vein 15, and the cuff 11 is positioned underneath the skin, as illustrated in FIG. 7C. It is contemplated that the catheter shaft 7 can be inserted into either the right or left internal jugular vein, but the right internal jugular vein is preferred, as also illustrated in FIGS. 4 and 5. In other non-limiting examples, the catheter shaft 7 may be inserted into the femoral vein or the subclavian vein. In another exemplary aspect, the catheter shaft 7 may be inserted into any vein of a patient. The placement of the small catheter tip 8 in a large blood vessel, such as the jugular vein, is beneficial because it decreases the rate of catheter occlusion, allows for high blood flow rates required for efficient dialysis, and allows for the rapid delivery and dilution of drugs into the bloodstream, thus providing more effective treatment. It is desired to accurately place the catheter tip 8 within the jugular vein 15 in order to make the catheter tip 8 less likely to contact or rest up against the inside of a blood vessel wall, which could block the catheter ports or cause damage to the vessel wall.

As the catheter 1 is tunneled into the insertion site 68, the circular in-growth cuff 11 that is attached to the outer surface 21 of the catheter shaft 7 distal of the bifurcation hub 49 is positioned inside of the incision site 68 underneath the skin, as illustrated in FIGS. 5A, 7C, and 7D. Because it takes approximately two weeks for tissue to become fully incorporated into the in-growth cuff 11, a medical practitioner typically fixes the catheter 1 to the patient's skin immediately after insertion. This position provides for optimal in-growth of the cuff 11 and secure fixation of the catheter 1. Proper catheter cuff placement also ensures a tight seal between the catheter 1 and the tunnel track, which reduces the risk of infection and associated complications. In addition, the position of the cuff 11 is important for patient comfort because the cuff 11 position determines where the catheter will be sutured to the patient's body.

The in-growth cuff 11 acts as an anchor to help secure the catheter in place and prevent movement of the catheter. The in-growth cuff 11 also helps to provide protection against infections related to vascular access catheters by sealing the tunnel track and, as such, reduces long term infection risks in the patient. In one aspect, the in-growth cuff 11 can be made of a porous or fibrous material such as Dacron® polyester and similar materials which promotes tissue in-growth by allowing the tunnel track tissue to grow into the in-growth cuff material. The in-growth cuff 11 prevents undesired movement and inadvertent removal of the catheter and thereby prevents excessive blood loss or death.

After the catheter 1 is fully inserted into the jugular vein, the guide wire 37 is removed. All of the lumens of the catheter 1 are aspirated (to ensure that they are all positioned inside the vein) and flushed. A chest x-ray is typically performed to ensure the catheter is positioned properly. The pre-attached suture 60 is positioned around the catheter hub 49, as illustrated in FIG. 7C, in grooves 72 or between rings 25a, 25b. Suture 60 is tied tightly around the grooves 72 in the hub 49, such that the catheter 1 is securely fixed to the patient's skin, as illustrated in FIG. 7D. Optionally, in another embodiment, the suture 60 can be tied around either extension tube 50 or 51 close to the bifurcate area of the catheter 1 (not shown). As illustrated in FIG. 7D, at least a portion of the catheter shaft 7 has been inserted into the patient's jugular vein, and the suture 60 has been pulled tight around the bifurcate 49 such that it rests in the grooves 72 of the bifurcate 49 or between rings 25a, 25b. The securement of the suture 60 to the bifurcate 49 anchors the catheter 1 very close to the insertion site, thereby preventing the catheter 1 from becoming dislodged. It also helps to maintain the position of the catheter 1, which is beneficial, especially for small cuffed catheters, such as the disclosed micro central line access catheter. The excess suture 60 can then be removed manually, or if the suture is bio-reabsorbable, the suture can be absorbed by the body.

In summary, the method of inserting the micro central line access catheter 1 can comprise one or more of: providing the catheter 1 described herein, inserting a guide wire 37 into a vessel in a patient body, particularly the internal jugular vein 15; inserting the catheter 1 over the proximal end of the guide wire 37; advancing the catheter 1 over the guide wire 37; inserting the catheter 1 into the internal jugular vein in a patient body over the guide wire 37; positioning the distal portion of the catheter 1 at a desired location within the jugular vein 15; and removing the guide wire 37 from the catheter 1. In one aspect, the method can further comprise the additional step of suturing the catheter to the patient's body, as described above.

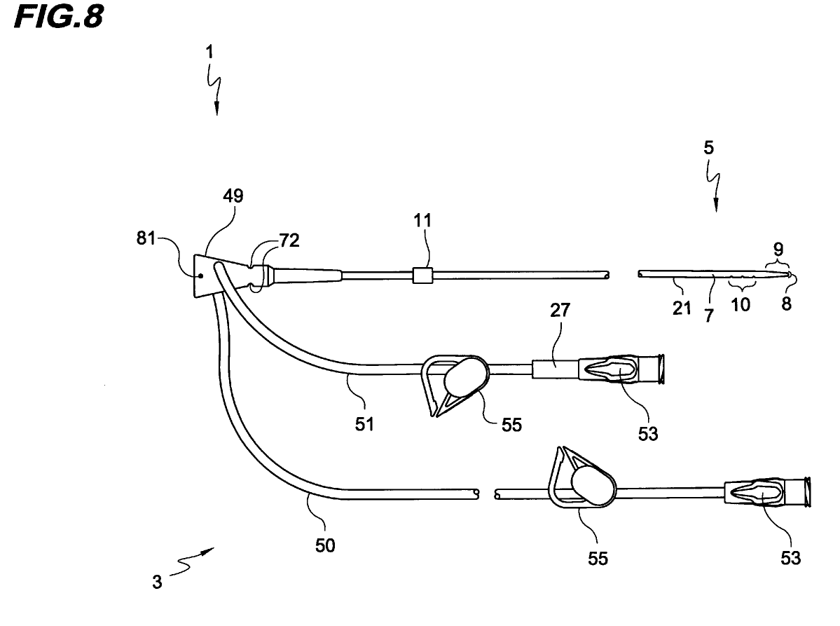

In one aspect, in another embodiment, the micro central line vascular access catheter can have curved extension tubes 50, 51, as illustrated in FIG. 8. In this aspect, at least a portion of a proximal portion of the extension tubes 50, 51 are disposed therein the outer surface of the hub 49 and extend outwardly toward the distal end of the catheter. In one aspect, the tubes 50, 51 extend outwardly toward one side of the catheter hub 49. This extension tube arrangement is beneficial because both of the extension tubes 50, 51 can be positioned away from the hub 49, the insertion site 68, and the patient's mouth. This extension tube position decreases the risk of infection. In this embodiment, as illustrated in FIG. 8, the hub 49 is streamlined and does not have any suture wings 49 to allow for the extension tubes 50, 51 to extend out of the hub 49. In one aspect, the hub may have grooves 72 defined therein the exterior surface of the hub 49, or it may have two rings 25a, 25b circumferentially surrounding the hub 49, as previously described.

As illustrated in FIGS. 1 and 4 through 10B, the hub 49 has an exterior surface. As illustrated in FIGS. 9A and 9B, the hub further defines an interior cavity that is connected to an opening 81 that is defined in the exterior surface of the hub. In one aspect, the hub 49 can have a self-sealing valve 19 that is mounted at least partially therein the interior cavity of the hub 49 and that extends at least to the opening 81 of the hub 49. In another aspect, at least a portion of the self-sealing valve 19 can extend outwardly away from the exterior surface of the hub 49 proximate the opening of the hub 49. In one aspect, as illustrated in FIG. 9B, the valve 19 can be defined therein the interior cavity of the hub 49 between the extension tubes 50, 51, such that the valve 19 is in fluid communication with at least one of the lumens of the catheter 1. In one exemplary aspect, valve 19 is in fluid communication with lumen 57 in the dual lumen configuration. Alternatively, in another embodiment, the valve 19 can be in fluid communication with lumen 73 in the single lumen configuration.

In one aspect, the valve 19 can be a duck bill valve 19 (such as supplied by Vernay Laboratories, Inc.), which is accessible through the opening 81 that is defined therein the exterior of the catheter hub 49. In this embodiment, the duck bill valve 19 is a compact valve 19 that acts a simple check valve in a small space within the cavity of the hub 49. In one aspect, the valve 19 can have a self-sealing diaphragm or opening defined on a surface of the valve 19. In another exemplary aspect, any other suitable type of valve can be used, such as, but not limited to, the Interlink® catheter system (Becton Dickinson & Co.).

As illustrated in FIGS. 9A and 9B, in one aspect, the micro central line access catheter 1 described herein can be used with a guide wire 37. In one aspect, the opening 81 can be configured to selectively receive a guide wire 37. As illustrated in FIG. 9A, guide wire 37 can be inserted through opening 81 and further advanced into one of the lumens of the micro central line vascular access catheter 1 through the self-sealing valve 19. The guide wire 37 can be inserted into opening 81 through the hub 49 of the catheter 1. As the guide wire 37 is inserted into the opening 81 through the duckbill valve 19, the opening 81 of the valve 19 self-seals around the guide wire 37. The small size of the catheter shaft 7 allows for easy insertion over an 0.018 inch guide wire which eliminates air embolization during insertion. In a typical procedure, a sheath could be used to insert the catheter into the jugular vein 15, but this can sometimes cause bleeding or can cause air to be drawn in to the catheter. The method of using the catheter 1 described herein can decrease the chance of air embolisms, by placing it over a guide wire 37, thereby preventing air from being drawn in, providing a self-dilating catheter, and eliminating the need for a sheath. FIG. 10A is a plan view of the micro central line vascular access catheter of FIG. 8 with a guide wire 37 inserted into lumen 57 of the catheter 1. FIG. 10B is a plan view of the micro central line vascular access catheter of FIG. 8 with a guide wire 37 inserted into the lumen 73 of the single lumen catheter embodiment. It is contemplated that the guide wire 37 can be used in the same manner in each of the catheter embodiments described herein.

In one aspect, as the guide wire 37 is removed from the hub 49, it is removed proximal to the cuff 11. When the guide wire 37 is pulled out of the catheter 1, the opening 81 self-seals. In one aspect, the guide wire 37 can be removed from the side of the catheter 1, or the guide wire 37 could be downwardly angled between extension tubes 50, 51. After the guide wire 37 is removed from the valve 19, the opening 81 of the valve 19 re-seals upon itself such that it forms a leak-proof seal. The opening 81 of the valve 19 of the catheter 1 disclosed herein is beneficial because it allows for placement of the catheter 1 over a guide wire 37 or stylet (not shown) to reduce the chance of air embolization.

In one aspect, a method of injecting an infusate into the catheter is provided. This method further comprises providing an infusate and injecting the infusate through at least one lumen of the catheter 1 and into a patient's body. In one aspect, the infusate can be selected from the group consisting of at least one of anti-restenosis agents, anti-thrombogenic agents, anti-inflammatory agents, anti-thrombotic agents, saline, contrast agents, urokinase, streptokinase, tissue plasminogen activator (t-PA), fibrinolytic agents, anti-proliferative agents, chemotherapeutics, anti-coagulants and the like. In another aspect, the method can further include injecting infusates, such as contrast agents, under high pressure into at least one of the catheter lumens and into the patient's body for computed tomography (CT).

The micro central catheter line access catheter disclosed herein is beneficial because it functions like a PICC, yet it does not have the disadvantages of a PICC, including long procedure times and patient discomfort due to accessing small tortuous veins, such as the antecubital vein, which is especially problematic in obese patients. The shorter elongate working length of the catheter shaft 7 (i.e., 10 to 15 cm), and the smaller diameter of the catheter shaft 7 are beneficial because the catheter does not destroy small veins, compared to PICCs and other typical, relatively long and large catheter shafts that are positioned in very small veins that can result in clotting and destruction of the vein, including the subclavian vein. Such long and large catheters typically have small internal diameters which only permit low flow rates of, for example, 5 cc/sec, which is sub-optimal for dialysis, pheresis, and CTA (computed tomography angiography). The catheter disclosed herein is also beneficial because it may have two lumens which are needed for dialysis and pheresis.

The catheter disclosed herein also overcomes the deficiencies of a PICC by eliminating the potential pain that patients can experience when they are being subject to multiple needle sticks using a standard peripheral PICC line, especially in obese patients and patients without patent peripherial veins. The catheter disclosed herein requires only one needle stick into the jugular vein of the catheter, followed by insertion of the catheter into the jugular vein, instead of multiple insertions during short periods of time, which can destroy the veins over time. Thus, insertion of the micro central line catheter requires minimal procedure time, for example, no more than two minutes, to insert the catheter into the vein under ultrasound, such as the internal jugular vein, which reduces the chance of clotting and destroying the vein or other complications.

The micro central line access catheter disclosed herein is beneficial because, in addition to overcoming several disadvantages of a PICC, it also overcomes the disadvantages of central venous catheters. Currently, central venous catheters (CVCs) are typically placed into the vein of a patient for various procedures using large 12-14 French tunneled catheters. However, the insertion into the vein using these large catheters can be painful to the patient, can cause long procedure times, and can also cause an increased risk of infection to a patient. The micro central line catheter disclosed herein overcomes these deficiencies by providing a catheter with a small catheter shaft 7 that is much smaller compared to a typical central line and is configured to enter the large vein, such as the internal jugular vein, or other veins, with minimal discomfort. Thus, the micro central line access catheter disclosed herein is easy for a medical practitioner to place into the vein, and functions similar to a central venous catheter (CVC).

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. The words “including” and “having,” as used herein including the claims, shall have the same meaning as the word “comprising.” Those familiar with the art can recognize other equivalents to the specific embodiments described herein, which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the selected embodiments of the invention. Those skilled in the art can recognize other equivalents to the specific embodiments described herein which equivalents are intended to be encompassed by the claims attached hereto.

1. A vascular access catheter, comprising:
a catheter shaft configured for insertion into a vein of a patient, comprising:
an exterior surface extending therebetween a proximal end and an opposed distal end, wherein the exterior surface of the catheter shaft further defines a plurality of openings positioned therein the distal end portion of the catheter shaft;
a first lumen extending from the proximal end to the distal end portion of the catheter shaft, wherein the first lumen is in fluid communication with at least one opening of the plurality of openings;
a second lumen extending from the proximal end to the distal end portion of the catheter shaft;
a first extension tube and a second extension tube, wherein the first extension tube is positioned in fluid communication with the first lumen and the second extension tube is positioned in fluid communication with the second lumen, wherein the second extension tube is configured for high pressure CT injections, and wherein the first extension tube is longer in length than the second extension tube.

2. The catheter of claim 1, wherein the at least one opening of the plurality of openings that is in fluid communication with the first lumen is defined therein a sidewall of the distal end portion of the catheter shaft.

3. The catheter of claim 2, wherein the at least one opening of the plurality of openings that is in fluid communication with the first lumen comprises a plurality of first lumen openings.

4. The catheter of claim 3, wherein the plurality of first lumen openings are positioned parallel to a longitudinal axis of the catheter shaft.

5. The catheter of claim 1, wherein the at least one opening of the plurality of openings that is in fluid communication with the first lumen is defined therein at the distal end of the catheter shaft.

6. The catheter of claim 1, wherein the at least one opening of the plurality of openings that is in fluid communication with the first lumen is spaced from the at least one opening of the plurality of openings that is in fluid communication with the first lumen.

7. The catheter of claim 1, wherein the catheter comprises:
a hub having an exterior surface, the hub further defining an interior cavity and a opening in the exterior surface of the hub, wherein the interior cavity is connected to the opening; and
a self-sealing valve mounted at least partially therein the interior cavity of the hub and extending at least to the opening of the hub.

8. The catheter of claim 7, wherein at least a portion of the self-sealing valve extends outwardly away from the exterior surface of the hub proximate the opening of the hub.

9. The catheter of claim 7, wherein the self-sealing valve is configured for selective receipt of a guide wire.

10. The catheter of claim 7, wherein the hub comprises at least one groove defined therein the exterior surface of the catheter hub.

11. The catheter of claim 10, wherein the at least one groove is configured for selective receipt of at least one suture.

12. A vascular access catheter, wherein the catheter shaft is configured for insertion into the internal jugular vein of a patient,

13. The catheter of claim 1, wherein the catheter comprises:
a hub having an exterior surface, wherein at least two rings substantially circumferentially surround at least a portion of the exterior surface of the hub, and wherein the rings are positioned substantially adjacent to one another and are configured for selective receipt of a suture.

14. The catheter of claim 1, further comprising a cuff that is mounted to a portion of the exterior surface of the shaft.

15. The catheter of claim 14, wherein the cuff substantially circumferentially surrounds the portion of the exterior surface of the catheter shaft.

16. The catheter of claim 14, wherein at least a portion of an inner surface of the cuff is adhered to at least one suture.

17. A vascular access catheter comprising:
a catheter shaft comprising an exterior surface extending therebetween a proximal end and an opposed distal end;
a cuff that is mounted to a portion of the exterior surface of the shaft, wherein the cuff substantially circumferentially surrounds the portion of the exterior surface of the catheter shaft, and wherein at least a portion of an inner surface of the cuff is adhered to at least one suture.

18. A vascular access catheter, comprising:
a catheter shaft configured for insertion into a vein of a patient, comprising:
an exterior surface extending therebetween a proximal end and an opposed distal end, wherein the exterior surface of the catheter shaft further defines a plurality of openings positioned therein the distal end portion of the catheter shaft;
a catheter lumen extending from the proximal end to the distal end portion of the catheter shaft; wherein the catheter lumen is in fluid communication with at least one opening of the plurality of openings;
a first extension tube and a second extension tube, wherein the first extension tube is positioned in fluid communication with the first lumen and the second extension tube is positioned in fluid communication with the second lumen, wherein the second extension tube is configured for high pressure CT injections, and wherein the first extension tube is longer in length than the second extension tube.

19. A method of inserting a catheter into a patient body, comprising:
providing a catheter, wherein the catheter is a vascular access catheter, comprising:
a catheter shaft configured for insertion into a vein of a patient, comprising:
an exterior surface extending therebetween a proximal end and an opposed distal end, wherein the exterior surface of the catheter shaft further defines a plurality of openings positioned therein the distal end portion of the catheter shaft;
a first lumen extending from the proximal end to the distal end portion of the catheter shaft, wherein the first lumen is in fluid communication with at least one opening of the plurality of openings;
a second lumen extending from the proximal end to the distal end portion of the catheter shaft;
a first extension tube and a second extension tube, wherein the first extension tube is positioned in fluid communication with the first lumen and the second extension tube is positioned in fluid communication with the second lumen, wherein the second extension tube is configured for high pressure CT injections, and wherein the first extension tube is longer in length than the second extension tube;
inserting a guide wire into a vein of a patient;
inserting the proximal end of the guide wire into the catheter;
advancing the catheter over the guide wire;
inserting the catheter into the vein over the guide wire;
positioning the distal portion of the catheter at a desired location within the vein; and
removing the guide wire from the catheter.

20. A method of injecting an infusate into a patient body, wherein the method comprises:
providing a catheter, wherein the catheter is a vascular access catheter comprising:
a catheter shaft configured for insertion into a vein of a patient, comprising:
an exterior surface extending therebetween a proximal end and an opposed distal end, wherein the exterior surface of the catheter shaft further defines a plurality of openings positioned therein the distal end portion of the catheter shaft;
a first lumen extending from the proximal end to the distal end portion of the catheter shaft, wherein the first lumen is in fluid communication with at least one opening of the plurality of openings;
a second lumen extending from the proximal end to the distal end portion of the catheter shaft;
a first extension tube and a second extension tube, wherein the first extension tube is positioned in fluid communication with the first lumen and the second extension tube is positioned in fluid communication with the second lumen, wherein the second extension tube is configured for high pressure CT injections, and wherein the first extension tube is longer in length than the second extension tube;
inserting at least a portion of the vascular access catheter into the patient's body;
providing an infusate; and
injecting the infusate into at least one of the first or second extension tubes, into at least of the first lumen or the second lumen, and into the patient's body.

21. The method of claim 20, wherein the method further comprises injecting the infusate into the second extension tube into the second lumen.

22. The method of claim 20, wherein the method further comprises injecting the infusate under high pressure.
